(19) United States
(12) Patent Application Publication
(10) Pub. No.: US 2024/0329024 A1
(43) Pub. Date: Oct. 3, 2024

(54) SYSTEM AND METHOD FOR CALIBRATION OF INACCESSIBLE REMOTE GAS HVAC SENSORS

(71) Applicant: SAUDI ARABIAN OIL COMPANY, Dhahran (SA)

(72) Inventor: Ibrahim S. AL RASHED, Dhahran (SA)

(73) Assignee: SAUDI ARABIAN OIL COMPANY, Dhahran (SA)

(21) Appl. No.: 18/191,793

(22) Filed: Mar. 28, 2023

(51) Int. Cl. G01N 33/00; F24F 11/49; F24F 11/89

(52) U.S. Cl. CPC G01N 33/007; F24F 11/49; F24F 11/89; G01N 33/0006; F24F 2110/65; G01N 33/0072

(57) ABSTRACT

A system for servicing a gas sensor disposed at a first location in an HVAC installation includes a service unit coupleable to the sensor and disposed at a second location, and a conduit configured to deliver test gas from the second location to a detector of the sensor. The service unit is operable to communicate bidirectionally with the sensor to exchange information therewith in conjunction with delivery of test gas through the conduit to the detector.

### FIELD OF THE DISCLOSURE

The present disclosure relates generally to gas sensors in heating and ventilation air conditioning (HVAC) systems, and more particularly, to the remote calibration of gas sensors that are disposed in inaccessible locations.

### BACKGROUND OF THE DISCLOSURE

Heat and ventilation air conditioning systems (HVAC) that are found in industrial environments may be particularly vulnerable to gas leaks from industrial operations. Dangerous gases can inadvertently enter the HVAC systems through ducts, joints, connectors, equipment and so forth. The gas leaks may lead to the spread of gases throughout a plant, posing health risks to personnel and, in the case of flammable or reactive gases, risking explosion and grave destruction and bodily injury.

Gas leak detection is an important part of HVAC operation. Gas sensors can be disposed in various locations in an HVAC system, to detect dangerous gases and provide warnings of their presence and/or concentration. Conventionally, for example, HVAC rooms may be equipped with LEL/H2S (lower explosive limit/hydrogen sulfide) sensors that are installed in air intake ducts to protect an air conditioning system from unexpected gas leaks that may happen and cause explosion or undesired incidents. The LEL/H2S sensors require preventative maintenance and calibration on a regular basis because of degradation due to normal wear that may be exacerbated by the harsh industrial environments of the HVAC system. However, the sensors may often be in difficult to access locations, and significant time and effort may be required to perform the regularly-required maintenance and calibration. For example, workers may need to climb scaffolding and brave sharp seams and edges in the ductwork to gain access to a sensor and conduct the necessary testing, which in some cases may require dismounting it and then remounting it after testing and calibration under difficult and unsafe conditions.

There is a long-felt need to mitigate the problem of inaccessibility of HVAC sensors for testing and calibration and performance of regular preventative maintenance. Such mitigation would reduce the maintenance cost and the risks to personnel charged with such maintenance, and would facilitate compliance with regular maintenance schedules, improving overall plant safety and minimizing health risks, material damage and even catastrophic destruction and failure.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system for servicing a gas sensor disposed at a first location in an HVAC installation includes a service unit coupleable to the sensor and disposed at a second location, and a conduit configured to deliver test gas from the second location to a detector of the sensor. The service unit is operable to communicate bidirectionally with the sensor to exchange information therewith in conjunction with delivery of test gas through the conduit to the detector.

In another embodiment, a method for servicing a gas sensor of an HVAC, the gas sensor having a detector operable to detect a gas of interest at a first location, includes establishing a connection between the gas sensor and a service unit disposed at a second location, exchanging information bidirectionally between the gas sensor and the service unit, and delivering test gas from the second location to the gas sensor while exchanging said information.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a schematic diagram of a remote gas sensor servicing system in accordance with certain embodiments.

FIG. 2 is a flow diagram of a method for servicing a sensor in accordance with certain embodiments.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to gas sensors in HVAC systems, and more particularly, to the remote calibration of gas sensors that are disposed in inaccessible locations.

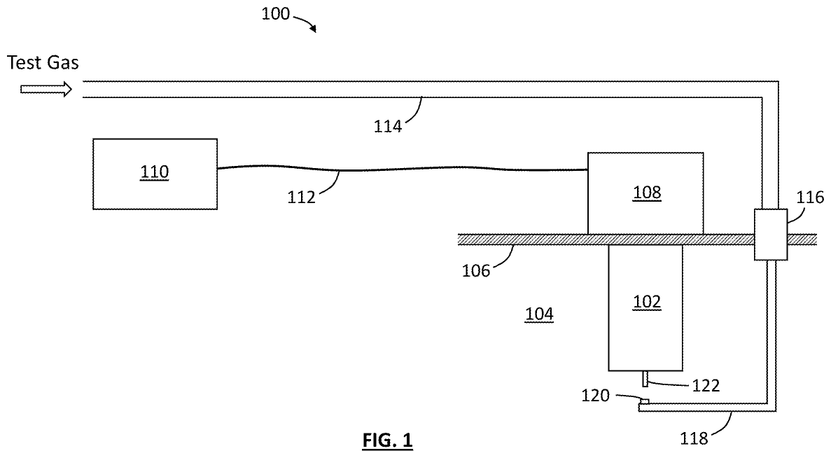

FIG. 1 is a schematic diagram of a remote gas sensor servicing system 100 in accordance with certain embodiments. An existing sensor 102 may be mounted in an interior region 104 of an HVAC installation, such as a duct that is bounded by a duct wall 106. The existing sensor 102 can be at a location that is difficult or dangerous for personnel to access, such as in an upper corner of a chamber, and tens of feet above ground in a cramped space behind other equipment (not shown). For this reason, the sensor interface 108, for example a junction box with appropriate connections and other outputs, such as a user interface (not shown) located exteriorly of the duct, may be electrically coupled to a service unit 110, for example by way of an electrical cable 112, that is disposed at a more accessible location, for example in the same chamber or a different chamber, but at a non-cramped or lower location. An alternative, wireless connection (not shown) between the sensor interface 108 and service unit 110 is also contemplated. It is also contemplated that that service unit 110 is portable or handheld, rather than being at a fixed location. In certain embodiments, it may be a smart phone or other personal electronic device, or a laptop or the like, and may be equipped with a suitable program or “app” (application) to conduct the desired testing and/or calibration.

System 100 also includes a hose or tubing 114, communicatively coupled, by way of a fitting 116, to a hose or pipe 118 having a nozzle opening 120 at one end thereof. Together, tubing 114 and hose or pipe 118 form a test gas delivery conduit to the sensor 102. The nozzle opening 120 is disposed in close proximity to detector 122 of sensor 102, and is operable to deliver the test gas, such as sulfur dioxide, to the sensor during a sensor servicing operation. The servicing operation, it will be appreciated, can refer to any adjustment, routine or unscheduled maintenance, or calibration of the sensor 102, and entails delivery of test gas in conjunction with receipt of readings and indications from the sensor 102 and/or sensor interface 108 by way of the service unit 110, and delivery of commands and data from the service unit 110 to the sensor 102 by way of the interface. In certain embodiments, however, the interface 108 may be omitted and communication may be directly between the service unit and the sensor 102. The servicing is conducted by an operator (not shown) from a more accessible location than that of the sensor 102. While in the present example the service unit 110 (and operator) are on opposite sides of duct wall 106 from sensor 102, this is not by way of limitation, and in some embodiments the service unit 110 and sensor are on the same side as the duct wall 106—that is, both can be outside the duct or inside the duct.

Tubing 114 may be mountable to the fitting 116 in a removable configuration, and may be installed during a testing/calibration operation, whereas hose or pipe 118 may be fixed in position in the interior region 104 of the duct. Hose or pipe 118 as configured is sufficiently spaced from the detector 122 to avoid obstructing its receipt of gases of interest during normal operation. In certain embodiments, fitting 116 may be sufficiently spaced from the sensor 102 and interface 108 so as to also be disposed at a more accessible location than the sensor and interface. In such an arrangement, the configuration of hose or pipe 118 would be appropriately changed—for example lengthened or differently-shaped—to be able to effectively deliver the test gas during testing/calibration operations. Such a configuration would be more amendable to allowing a removable tubing 114, which the operator can bring to the accessible fitting to conduct the servicing process, and then remove and take away after the servicing.

The system 100 is operable to extend the reach of operators performing sensor maintenance and calibration, keeping them out of harm's way while maintaining the sensor 102 in good service order. The operator can, from the safe and accessible location of the calibration unit 110, deliver a test gas through the tubing 114 and monitor and adjust the operation of the sensor 102. Service unit 110 is configured to provide indications of the operation of the sensor 102, through the user interface, during the servincing procedure, and to permit the operator to enter and/or change operating parameters, sensitivities, etc., of the sensor as necessary to bring it to good working order. As mentioned above, service unit 110 may be at a fixed location and permanently coupled (wired or wirelessly) to the sensor 102 by way of the interface 108 for example, or it may be a handheld device or the like that is temporarily connected thereto for the testing/calibration operation.

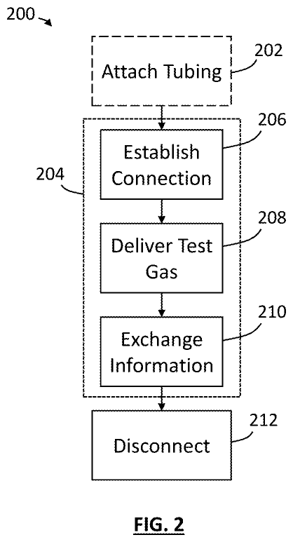

FIG. 2 is a flow diagram of a method 200 for servicing a sensor such as sensor 102 in accordance with certain embodiments. The method begins at 202 with the operator attaching the tubing 114 to the fitting 116 if it is not already attached. The broken lines indicate that this is an optional step conducted if the tubing is not already attached to the fitting in a more permanent configuration that is also contemplated herein. At 204 the servicing process is commenced, with an electronic connection established at 206 between sensor interface 108 and service unit 110, so that information can be exchanged bidirectionally between the service unit 110 and the sensor 102. The exchanged information can include readings, measurements and status information from the sensor 102, and commands and information from the service unit 110 to adjust operation of the sensor, for example for calibration, resetting, or other purposes. The electronic connection may be established by way of plugging in one or both ends of the cable 112 into the respective device and/or initiating a wired or wireless communication protocol. At 208, test gas is delivered to the sensor by way of tubing 114, fitting 116, hose or pipe 118, and nozzle 120 as explained above. At 210, readings and other information from sensor 102 are obtained by the operator for example in conjunction with gas delivery, and via the service unit 110; and any necessary calibration and adjustment actions of the senor 102 are performed, again by way of the user interface of the service unit 110. This step, and optionally the gas delivery step, may be performed several times as necessary to bring the sensor 102 into good working order. At 212, when it is ascertained that the sensor 102 has been properly serviced and calibrated, the service unit 110, and optionally the tubing 114, are disconnected, and the process is deemed completed. Using the above servicing procedure ensures that the sensor 102 need not be disassembled or disturbed, or the duct opened. The sensor remains in place and the duct remains closed throughout the operation, more faithfully mimicking the operating environment. Conventionally, by contrast, such disturbance and duct access leads to inaccuracies and undesirable fluctuating readings, making calibration more difficult.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A system for servicing a gas sensor having a detector operable to detect a gas of interest at a first location in an interior of a heating and ventilation air conditioning system (HVAC) installation, the system comprising:
a service unit coupleable to the sensor and disposed at a second location exterior of the HVAC installation; and
a conduit configured to deliver test gas from the second location to the detector of the sensor,
wherein the service unit is operable to communicate bidirectionally with the sensor to exchange information therewith in conjunction with delivery of test gas through the conduit to the detector.

2. The system of claim 1, wherein the gas sensor is a LEL/H2S (lower explosive limit/hydrogen sulfide) sensor.

3. The system of claim 1, wherein the conduit comprises a tubing disposed exterior of the HVAC installation, a pipe disposed interior of the HVAC installation, and a fitting coupling the tubing to the pipe and mounted in a wall separating the exterior from the interior.

4. The system of claim 3, wherein the tubing is attached to the fitting in a removable configuration.

5. The system of claim 3, wherein the pipe is mounted to the fitting in a fixed configuration.

6. The system of claim 1, wherein the information includes one or more of readings, measurements and status information from the sensor.

7. The system of claim 1, wherein the information includes commands from the service unit to adjust operation of the sensor.

8. A method for servicing a gas sensor of a heating and ventilation air conditioning system (HVAC), the gas sensor having a detector operable to detect a gas of interest at a first location, the method comprising:
establishing a connection between the gas sensor and a service unit disposed at a second location;
exchanging information bidirectionally between the gas sensor and the service unit; and
delivering test gas from the second location to the gas sensor while exchanging said information.

9. The method of claim 8, wherein the gas sensor is a LEL/H2S (lower explosive limit/hydrogen sulfide) sensor.

10. The method of claim 8, wherein the information includes one or more of readings, measurements and status information from the sensor.

11. The method of claim 8, wherein the information includes commands from the service unit to adjust operation of the sensor.

12. The method of claim 8, wherein the one of the first and second locations is interior of the HVAC, and the other of the first and second locations is exterior of the HVAC.

13. A system for servicing a gas sensor disposed at a first location in a heating and ventilation air conditioning system (HVAC) installation, the system comprising:
a service unit coupleable to the sensor and disposed at a second location; and
a conduit configured to deliver test gas from the second location to a detector of the sensor,
wherein the service unit is operable to communicate bidirectionally with the sensor to exchange information therewith in conjunction with delivery of test gas through the conduit to the detector.

14. The system of claim 13, wherein the information includes one or more of readings, measurements and status information from the sensor.

15. The system of claim 13, wherein the information includes commands from the service unit to adjust operation of the sensor.
